(19) United States
(12) Patent Application Publication
(10) Pub. No.: US 2026/0106023 A1
(43) Pub. Date: Apr. 16, 2026

(54) METHOD, DEVICE AND COMPUTER-READABLE MEDIA FOR TRANSMITTING INFORMATION FROM A MEDICAL DEVICE TO A SERVER

(71) Applicant: Maquet Critical Care AB, Solna (SE)

(72) Inventor: Jonas ANDERSSON, Tyresö (SE)

(21) Appl. No.: 19/422,137

(22) Filed: Dec. 16, 2025

Related U.S. Application Data
(63) Continuation of application No. PCT/EP2024/067822, filed on Jun. 25, 2024.

(30) Foreign Application Priority Data
Jun. 27, 2023 (SE) 2350794-0

(51) Int. Cl. G16H 40/67; G06F 21/62; G16H 10/60

(52) U.S. Cl. CPC G16H 40/67; G06F 21/6245; G16H 10/60

(57) ABSTRACT

The present disclosure relates to a method for transmitting information from a medical device to a first server, the method comprising: receiving first access level data, the first access level data indicating whether the first server is configured to receive personal identifiable information, PII, or not from the medical device; determining available information at the medical device; classifying the available information into at least one class of a plurality of classes, the plurality of classes comprising PII and non-PII; upon the first access level data indicating that the first server is not configured to receive PII, filtering the available information to remove PII and transmitting the filtered available information from the medical device to the first server; and upon the first access level data indicating that the first server is configured to receive PII, transmitting the available information from the medical device to the first server.

### CROSS-REFERENCE TO RELATED APPLICATIONS

This application is a continuation under 35 U.S.C. § 120 of International Application No. PCT/EP2024/067822, filed Jun. 25, 2024, which claims priority to Swedish Application No. 2350794-0, filed Jun. 27, 2023, under 35 U.S.C. § 119(a). Each of the above-referenced patent applications is incorporated by reference in its entirety.

### TECHNICAL FIELD

The present invention relates to medical device connectivity, and more specifically to technologies for transmitting information from the medical device to a server.

### BACKGROUND

Medical device connectivity has become an increasingly important aspect of healthcare technology, primarily due to the evolving need for real-time transfer and monitoring of patient data as well as equipment data. Medical devices, such as monitors, ventilators, extracorporeal membrane oxygenation devices, infusion pumps, and similar devices, may establish and maintain a connection through which data are transferred to a server, for example in a local or external device and data management (DDM) setting. The data transfer can be employed for various applications, including applications for patient monitoring, diagnostics, device status monitoring, and device control.

Personal identifiable information (PII), such as patient or clinical data, may be included in data obtainable from a medical device. This transfer of information can be subject to various data protection regulations that mandate the lawful, fair, and transparent processing of PII. These regulations often impose geographical limitations on PII data transfer and stipulate robust security measures, including requirements for encryption, pseudonymization, confidentiality, and the continuous integrity and resilience of data processing systems and services.

When data is stored externally, for instance, on a cloud-based server or a third-party bare metal server, it may be challenging to maintain comprehensive knowledge of how the data is processed, stored, and managed. Furthermore, controlling the physical location of the data might not always be feasible. These factors make compliance with the aforementioned data protection regulations potentially more challenging in such settings.

There is thus a need for improvements in this context.

### SUMMARY

In view of the above, solving or at least reducing one or several of the drawbacks discussed above would be beneficial, as set forth in the attached independent patent claims.

According to a first aspect of the present invention, there is provided a method for transmitting information from a medical device to a first server, the method comprising: receiving first access level data, the first access level data indicating whether the first server is configured to receive personal identifiable information, PII, or not from the medical device; determining available information at the medical device; classifying the available information into at least one class of a plurality of classes, the plurality of classes comprising PII and non-PII; upon the first access level data indicating that the first server is not configured to receive PII, filtering the available information to remove PII and transmitting the filtered available information from the medical device to the first server; and upon the first access level data indicating that the first server is configured to receive PII, transmitting the available information from the medical device to the first server.

The information obtainable from a medical device (i.e., the available information) typically varies based on the intended use and features of the medical device. The available information may generally include elements like log files, physiological alarms, technical alarms, measured or computed values for a medical device, data trends, configurations, settings, and information about the current patient, among others. This information can be categorized into several classes, primarily differentiated based on whether the data can be identified as Personal Identifiable Information (PII) or non-PII. Further sub-classes may also be formed to achieve a more granular classification of the information. The classes and/or sub-classes may be assigned to different roles or role categories implemented by the medical device and/or the first server.

PII may be understood as data that can be used on its own or with other information to identify a patient and typically include patient identification information such as name, ID number and the like. The PII may further relate to treatment information, such as oxygen concentration, tidal volume, blood flow rate, physiological responses, administered medications, and duration of the treatment. Non-PII information generally refers to information that cannot be used to identify, directly or indirectly, an individual, such as aggregate statistics, device information relating to make, model, software version, maintenance logs, and general usage information such as number of times a particular device has been used over a period of time. The non-PII information may also include de-identified data from which all identifying information has been removed, such as oxygen levels, heart rates, or blood pressure readings that are not tied to a specific individual.

The inventors have recognized that by establishing a communication protocol between the medical device and the primary server in accordance with the first aspect, the control of data flows for different information classes may be achieved automatically and with low complexity.

In examples, the first access level data may indicate the role categories that the first server implements. In other examples, the first access level data may specifically state which of the plurality of classes of information that the first server is configured to receive (or not configured to receive).

By the term “filtering the available information” should, in the context of present specification, be understood selecting the information among the available information that the first server is configured to receive according to the first access level data. For example, in a role-based scenario, the first access level data may indicate which role categories the first server implements. These role categories may be compared to the role categories that the medical device implements (i.e., the categories of the role(s) providing the available information). The information provided by the roles that overlaps with the roles of the first server may then be selected and transmitted to the first server. Generally, in case the first access level indicates that the first server is not configured to receive PII, any PII is removed (filtered, unselected, etc.) from the information to be transmitted. In the case the first access level indicates that the first server is configured to receive PII, the full available information may be transmitted.

Typically, the available information comprises both PII and non-PII. In some cases (e.g., in some settings of the medical device, or at some points in time, etc.), the available information may comprise only PII or only non-PII. In the case the available information comprises only PII, and the first server is not configured to receive PII, the medical device may transmit an empty message to the first server, or a message defining that no information is available for transmission. In the case the available information comprises only non-PII, the step of filtering the available information will not result in any removal of information from the available information before transmission to the first server.

In some embodiments, the non-PII comprises equipment data pertaining to the medical device. For example, non-PII may comprise information that enables monitoring of equipment performance, digitize best practices for equipment maintenance and reduce administration time. Advantageously, such information may facilitate easy service and quicker troubleshooting. By not limiting access to such information at the first server, improved asset management may be achieved. The non-PII may be displayed at a User interface (UI) of the first server.

In examples, the PII comprises clinical data pertaining to the medical device. Such information may facilitate improved surveillance of a patient currently treated or monitored by the medical device. Moreover, the PII may be utilized by one or more applications run in the application environment on the first server. The applications may, for example, be clinical applications or applications for decision support.

In some embodiments, medical device and the first server are arranged in a local network, wherein first access level data indicates that the server is configured to receive PII. Such local network may for example be a local area network (LAN) of a healthcare setting. Using a local server for data storage of PII and/or running clinical applications using the received PII can simplify adherence to various data regulations relating to PII as it provides clear control over data location, security measures, and access permissions. With a local server, awareness of exactly where the PII resides may be facilitated, eliminating concerns about international data transfers. Moreover, autonomy to implement tailored security measures may be achieved, as well as full transparency in understanding how data is being processed, stored, and managed.

In examples, the method may further comprise the steps of receiving the transmitted information at the first server, the transmitted information comprising PII; receiving second access level data, the second access level data indicating that a second server is not configured to receive PII; and filtering the received information to remove PII and transmitting the filtered received information from the first server to the second server. In example embodiments, the first server and the medical device are arranged in a local network, and wherein second server is arranged externally to the local network. The local (first) server may thus be in communication with a further (second) server on an external network. Such communication may for example be based on the HTTP- or FTP-protocol. The second server may for example be a cloud-based server or a bare metal server under the control of a third-party provider and having a public IP address accessible at the public internet. Such an external server can be leveraged to facilitate remote monitoring of the medical device, encompassing aspects such as equipment performance and maintenance, from any location where a user might be present. By removing the PII before transmitting information to the second server, data regulations as discussed above may be adhered to. The filtering/selection may be implemented as discussed above for the information available at the medical device, for example by comparing role categories implemented by the first and the second server.

In some embodiments, the first server comprises an environment for running a clinical application using the information received from the medical device. Example of such clinical application comprises hospital alarm management, therapy centric applications, digital twin applications, etc. The first sever may alternatively or additionally comprises an environment for running device management applications, providing functionality such as service and troubleshooting, remote support, asset management, cost control, etc. It may be beneficial to run the applications on the first server instead of on the medical devices, since the first server may have a higher computing capacity compared to the medical device. Moreover, access to the first server may be easier to achieve for a user, compared to direct access to the medical device.

In examples, the medical device is arranged in a local network, and the first server is arranged externally to the local network. In these examples, the access level data may indicate that the first server is not configured to receive PII. In examples, the medical device may be connected to either a local or external server and controlling data flows based thereon, as described herein. An increased flexibility may thus be achieved. The first server may thus be arranged outside the healthcare setting, i.e., outside any firewall protecting the local network in which the medical device resides. In some examples, the external server is provided on a cloud computing platform. The local server may comprise an environment for running applications assisting technical staff performing service and maintenance of the medical devices installed in a healthcare setting. This is also referred to as ‘fleet management’ and may utilise equipment data received from the medical device and/or local server.

In some embodiments, the step of receiving first access level data comprises performing a handshake procedure between the medical device and the first server. A handshake procedure may be a process by which two devices (i.e., the medical device and the first server) agree upon the parameters of their interaction, including the roles and capabilities each one supports. The handshake procedure may for example comprise a role (e.g., role category) identification phase, a role matching phase and an agreement phase, before the medical device sends data to the first server based on the agreed-upon overlapping roles. For instance, if both devices support a PII-role category, the medical device may send patient data (PII) to the first server. Such a handshake procedure may for example allow for dynamic identification and agreement of roles, enabling flexibility if the roles supported by each device/server change over time.

In some embodiments, the step of receiving first access level data comprises: receiving a certificate associated with the first server; verifying the certificate to determine validity of the certificate; and upon determining that the certificate is valid, extracting the first access level data from the certificate. Advantageously, certificates can provide a robust mechanism for authenticating the identity of the server at the medical device, enhancing security. Moreover, with certificates, the role categories and roles can be defined in advance, potentially simplifying and speeding up the connection setup.

In examples, the first access level data is received upon installation of the medical device. The first access level may, for example, be received in response to the medical device being turned on and connected to the first server for the first time. Receiving the first access level data may be initiated by an operator inputting a command, or automatically by the medical device upon connection to first server. It will however be appreciated that the first access level data may be received in response to other events than installation. The first access level data may, for example, be received in response to the medical device being introduced into a medical treatment area, or in response to the medical device being powered into an active power state.

In some embodiments, the medical device is at least one of: a cardiac support device, a respiratory support device, or a or monitoring device.

In some embodiments, the medical device comprises a display, and wherein the method further comprises the step of displaying the available information on the display. Advantageously, features such as real-time monitoring and immediate feedback may be facilitated. Moreover, user experience may be improved, and data redundancy may be provided.

According to a second aspect of the invention, the above object is achieved by a medical device comprising: one or more processors; and one or more non-transitory computer-readable media storing first computer executable instructions that, when executed by the one or more processors, cause the medical device to perform actions comprising: receiving first access level data, the first access level data indicating whether a first server is configured to receive personal identifiable information, PII, or not from the medical device; determining available information at the medical device; classifying the available information into at least one class of a plurality of classes, the plurality of classes comprising PII and non-PII; upon the first access level data indicating that the first server is not configured to receive PII, filtering the available information to remove PII and transmitting the filtered available information to the first server; and upon the first access level data indicating that the first server is configured to receive PII, transmitting the available information to the first server.

According to a second aspect of the invention, the above object is achieved by one or more non-transitory computer-readable media storing instructions executable by one or more processors, wherein the instructions, when executed, cause the one or more processors to perform operations comprising: receiving first access level data, the first access level data indicating whether a first server is configured to receive personal identifiable information, PII, or not from the medical device; determining available information at the medical device; classifying the available information into at least one class of a plurality of classes, the plurality of classes comprising PII and non-PII; upon the first access level data indicating that the first server is not configured to receive PII, filtering the available information to remove PII and transmitting the filtered available information to the first server; and upon the first access level data indicating that the first server is configured to receive PII, transmitting the available information to the first server.

The second and third aspects may generally have the same features and advantages as the first aspect. It is further noted that the disclosure relates to all possible combinations of features unless explicitly stated otherwise.

### BRIEF DESCRIPTION OF THE DRAWINGS

The above, as well as additional objects, features, and advantages of the present invention, will be better understood through the following illustrative and non-limiting detailed description of embodiments of the present disclosure, with reference to the appended drawings, where the same reference numerals will be used for similar elements, wherein:

FIG. 1 show a system with a medical device and a first server according to a first embodiment;

FIG. 2 show a system with a medical device and a first server according to a second embodiment;

FIG. 3 show a system with a medical device, a first server and a second server according to a third embodiment;

FIG. 4 shows a flow chart of a method for transmitting information from a medical device to a first server.

### DETAILED DESCRIPTION

Device-based data access refers to a system of permissions and restrictions where access to data is determined by the specific device being used. This approach can enhance security by allowing sensitive data to be accessed only from secure, trusted devices. The present disclosure relates to a protocol for achieving device-based data access in a low complexity and automatic fashion. Moreover, the present disclosure provides techniques for facilitating that personal identifiable information (PII) is handled according to relevant data regulations, such as the General Data Protection Regulation (GDPR) applicable in the European Union (EU) at the time of filing of this disclosure.

Generally, a medical device employed at a healthcare setting may gather or otherwise comprise at least two classes of data, PII and non-PII. In some embodiments, PII is referred to as clinical data, and non-PII is referred to as equipment data. The classes of data may pertain to a plurality of roles implemented by the medical device. Table 1 below shows by way of example equipment data roles, where all roles belong to a non-PII role category:

TABLE 1

| Role | Description |
|---|---|
| Logs | Log files. |
| Status | E.g., technical status, installed components, |
| | hardware details, and usage statistics. |
| Diagnostics | System checks, self-test, self-diagnosis, etc. |

Table 2 below shows by way of example clinical data roles, where all roles belong to a PII role category:

TABLE 2

| Role | Description |
|---|---|
| Alarms | E.g., alarms relating to the current patient. |
| Metrics | Values produced or measured by the medical |
| | device. |
| Patient metadata | Information about the current patient, e.g., |
| | patient id, weight, age, etc. |

PII may not be transmitted without restrictions due to several reasons. For example, privacy laws such as GDPR in the European Union, the California Consumer Privacy Act (CCPA) in California, USA, and others globally, may legally restrict the free transmission and processing of PII. These laws mandate that PII must be handled carefully to respect individuals' privacy rights. Moreover, PII may be a valuable target for cybercriminals, who can use it for identity theft, fraud, or other malicious activities. Therefore, PII may need to be transmitted and stored securely to protect against unauthorized access and breaches.

As described above in conjunction with Table 1 and Table 2, a medical device may gather or otherwise comprise various data, both PII and non-PII. PII may be understood as information that typically includes patient identification information, such as name, ID number and the like, as well as treatment information, such as oxygen concentration, tidal volume, blood flow rate, physiological responses, and other type of data that on its own, or with other information, can be used to identify an individual. Non-PII, on the other hand, typically relates to information that cannot be used to identify an individual. Examples of such information include equipment data like make and model, software versions, maintenance information, and aggregate statistics on device usage and the like.

Both PII and non-PII may be valuable to access from other devices than the medical device, such as a server. Such a server may comprise an application runtime environment, in which the data may be used for various applications providing real-time information to a user and assisting in analytics, reporting and maintenance of the medical devices. For example, PII may typically be used by a healthcare provider to monitor, control, and optimise the healthcare delivered to the patient. Non-PII may typically be used for service and troubleshooting, remote support, asset management, cost control, etc. Non-PII is generally considered less sensitive from a patient integrity point of view and may not be subject to the same restrictions as PII.

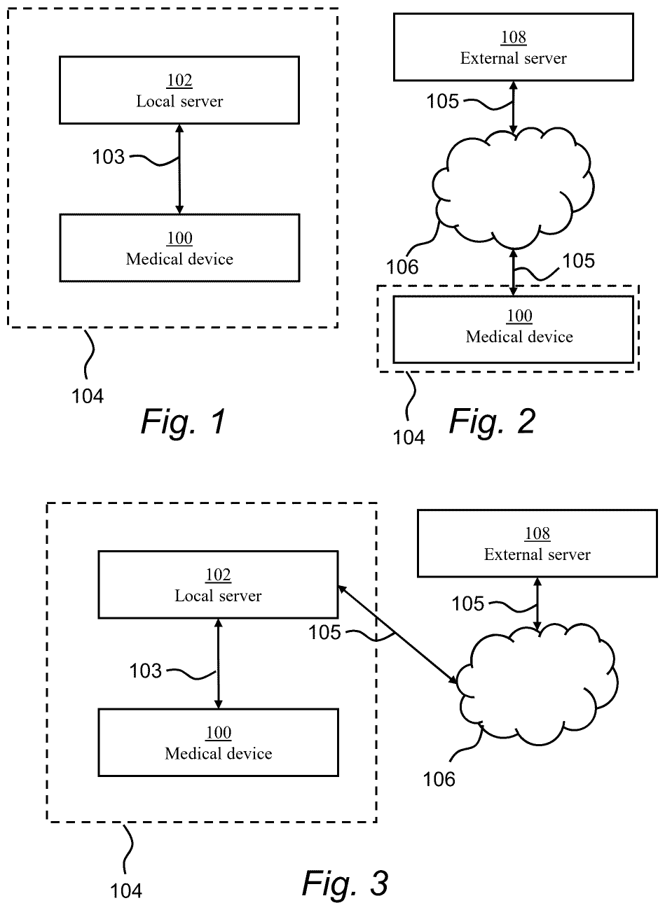

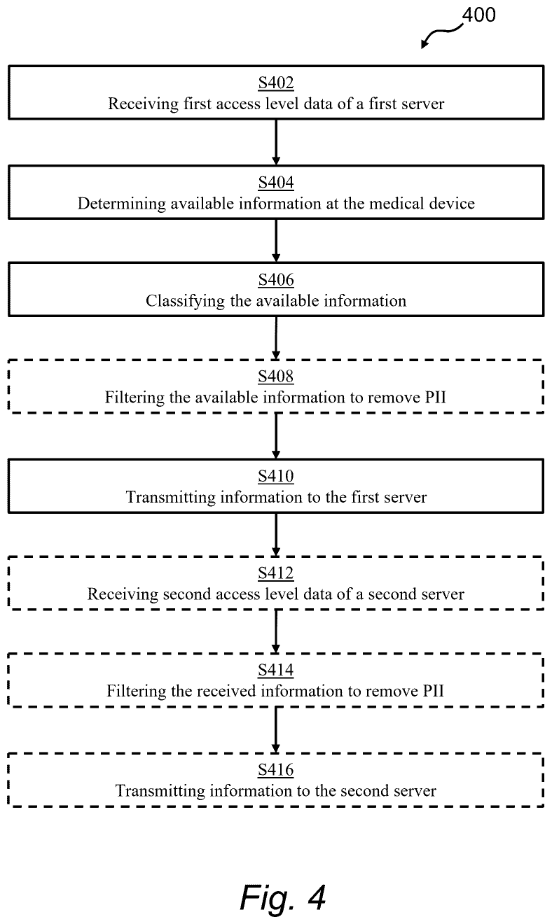

A medical device could be linked to and set up to relay information to both an internal and external server. As previously discussed, the manner in which data is transferred from the medical device to the server may require different handling, contingent upon whether the device is transmitting data to an external or internal entity. The upcoming discussion will detail a protocol designed to facilitate device-based data access in a low complexity and automated manner. This protocol applies to an array of system configurations depicted in FIGS. 1-3 and will be explored alongside the method illustrated in FIG. 4.

FIG. 1 shows by way of example a local server 102 according to an embodiment, comprising an environment for running an application that uses data from a medical device 100 connected to a local server 102. The local server 102 and the medical device 100 is arranged on a local network (local area network) 104 of a healthcare setting, such as a hospital. The communication within the local network may, for example, be wireless or wired. It should be noted that typically the local server 102 is connected to a plurality of medical devices, but for ease of explanation, FIG. 1 (and FIGS. 2-3 alike) only shows one medical device 100. The local server 102 may comprise an application runtime environment, comprising a container management platform such as a local Kubernetes platform. The environment may also be referred to as a Control Centre and may as discussed above form a platform for various applications providing real-time information to a user and assisting in analytics, reporting and maintenance of the connected devices.

The medical device 102 is thus connected to the local (first) server 102 through a wired or wireless connection 103. To setup the data restrictions that applies to this connection 103, the medical device 100 receives S402 a first access level data, the first access level data indicating whether the first server 102 is configured (allowed or not allowed) to receive PII or not from the medical device 100. Typically, in the setting of FIG. 1 where the first server 102 resides on the same local network as the medical device 100, the first access level data indicates that the first server is configured to receive PII. It should be noted that that in some embodiments, the medical device 100 receives the first access level data pertaining to the first server 102 from another entity than the first server 102, such as an access level coordinating device. In some embodiments, the first access level data is received upon installation of the medical device 100.

In one embodiment, the first access level data is received using a handshake procedure between the medical device 102 and first server 100. For example, the handshake procedure may comprise the following steps:

Initiation: The handshake begins when one device (usually the data provider, i.e., the medical device 100) sends a request to initiate communication to the other device (the data receiver, i.e., the first server 102).

Role Identification: Each device 100, 102 sends a list or signal indicating the roles or role categories it supports. For the data provider, this could be the types of data it can send, while for the data receiver, this could be the types of data it can process or handle.

Role Matching: The medical device 100 compare the role categories supported by the medical device 100 and the first server 102 and identify the overlapping role categories. Only the data associated with these overlapping role categories will be allowed to be sent from the provider to the receiver. This ensures that the receiver only gets the types of data it can handle.

Agreement: Once the overlapping role categories have been identified, the devices 100, 102 agree to communicate based on these role categories. This agreement completes the handshake, and data transmission can begin.

In this exemplary handshake procedure, the medical device 100 thus determines S404 available information at the medical device 100 and classifies S406 the available information into at least one class of a plurality of classes (put differently, at least one role category of a plurality of role categories), the plurality of classes comprising PII and non-PII. Put differently, the medical device 100 determines which role category/categories it support(s).

Finally, data is transmitted S410 from the medical device 100 to the first server 102 through the connection 103. In case the first access level data indicates that the first server is not configured to receive PII, a filtering step S408 is required to remove PII from the data to be transmitted. In the setting of FIG. 1, this step S408 is not needed and the medical device 100 may transmit all available information to the first server 102. In other words, in the terms of the handshake procedure exemplified above, the medical device 100 sends information to the first server 100 based on the agreed-upon role categories. For instance, if both devices support PII role(s) (see table 2 above), the medical device 100 can send PII to the first server 102.

In other embodiments, instead of a handshake procedure, step of receiving S402 first access level data is embodied by a certification process. For example, the medical device 100 may receive a certificate associated with the first server 102, verify that the certificate is valid and extracting the first access level data from the valid certificate.

By generalizing the first access level data to indicate if the if the first server is configured to receive PII or not, the complexity of the role matching and/or certificate process is reduced. For example, the first access level data may be determined based on whether the first server 102 is internal or external. An external server (as will be further described in FIGS. 2-3) may be controlled by an entity different from the one controlling the medical device 100 and the local server 102. An entity controlling an external server may, for instance, be a provider of the medical devices 100 (and, in some examples, the local server 102), and may not be configured to access PII. Moreover, as discussed above, the geographic location of an external server may not meet the requirements specified in privacy laws relating to PII, such that transmitting PII to an external server may violate such privacy laws.

FIG. 2 shows by way of example a setup where the first server is an external server 108. The external server 108 may comprise an environment for running an application that uses data from a medical device 100 connected 105 to the external server 108 via the public Internet 106. Both the medical device 100 and the external server 108 thus are assigned an IP (Internet Protocol) address, which uniquely identifies it on the Internet 106 such that data/information may be transmitted between the medical device 100 and the external server 108. In the embodiment of FIG. 2, the medical device 100 is thus arranged in a local network 104, and the first server is arranged externally to the local network 104.

Similar to as described in conjunction with FIG. 1 above, to setup data restrictions that applies to the connection 105 between the medical device 100 and the external server 108, the medical device 100 receives S402 first access level data, the first access level data indicating whether the external (first) server 108 is configured to receive PII or not from the medical device 100. Typically, in the setting of FIG. 2 where the first server 108 does not reside on the same local network 104 as the medical device 100, the first access level data indicates that the first server 108 is not configured to receive PII.

The step of receiving S402 the first access level data may be embodied by a handshake procedure, or a certification process as described above in conjunction with FIG. 1.

Since the first server 108 is external and thus connected 105 to the medical device 100 using the public internet, PII may not be allowed to be sent to the external server 108 from the medical device 100 for the reasons set out above. Consequently, before transmitting information from the medical device 100 to the external server 108, the information available at the medical device 100 is filtered S408 to remove PII. After that the filtered available information may be transmitted S410 from the medical device 100 to the external server 108. Transmission of such non-PII (e.g., equipment data) to an external server 108 may allow for technical staff to access and monitor data concerning the performance and status of the equipment (i.e., the medical device 100). The equipment data may, for example, be used on a global level to analyse and optimise various medical devices installed at various health care settings, without risking accessing the more sensitive PII. Further, the present example allows the external server 108 to access the non-PII without having direct access to the local network 104 in which the medical device 100 is installed.

In yet other embodiments, for example as shown in FIG. 3, the medical device is connected 103 to the local server 102 that is further connected 105, via the public internet 106, to the external server 108. In this embodiment, the information received at the local (first) server 102 may comprise PII which may need to be filtered before transmitting the filtered information (i.e., equipment data) to the second server 108. This process may be embodied similarly as described above and as shown in FIG. 4, e.g., by receiving S412, at the first (local) server 102 second access level data, the second access level data indicating that the second (external) server 108 is not configured to receive PII. The first server 102 may then filter S414 the received information from the medical device 100 to remove PII and transmit S416 the filtered received information from the first server 102 to the second server 108. Advantageously, the same protocol for setting up communication between any entity in systems like the ones described in FIG. 1-3 may be used.

It should be noted that although FIG. 4 shows the steps of the method as a sequence of successive steps, the steps need not be performed strictly in the shown order. Further, two or more steps may be performed simultaneously. For instance, access level may be received S402 after or between the steps of determining S404 and classifying S406 the available information at the medical device 100.

The medical device 100 in any of the settings in FIG. 1-3 may be any suitable medical device for treatment, or support of treatment, or a patient. In some embodiments, the medical device is one of a cardiac support device, a respiratory support device, or a or monitoring device. In some embodiments, the medical device 100 is further configured to display the available information on the display.

The medical device may generally comprise one or more processors and one or more non-transitory computer-readable media storing first computer executable instructions that, when executed by the one or more processors, cause the medical device to perform at least parts of the actions shown in FIG. 4 and described above.

Generally, the medical device 100 and the first/second server 102, 108 may comprise circuitry which is configured to implement (using one or more non-transitory computer-readable media) the functionality described herein. Suitable processors for the execution of a program of instructions include, by way of example, both general and special purpose microprocessors, and the sole processor or one of multiple processors or cores, of any kind of computer. The processors can be supplemented by, or incorporated in, ASICs (application-specific integrated circuits). Those skilled in the art will understand that the above-described exemplary embodiments may be implemented in any suitable software, hardware, or firmware configuration or combination thereof. An exemplary hardware platform for implementing the exemplary embodiments may include, for example, an Intel x86 based platform with compatible operating system, a Windows OS, a Mac platform and MAC OS, a mobile device having an operating system such as iOS, Android, etc. In a further example, the exemplary embodiments of the above described method may be embodied as a program containing lines of code stored on a non-transitory computer readable storage medium that, when compiled, may be executed on a processor or microprocessor.

Additionally, variations to the disclosed embodiments can be understood and effected by the skilled person in practising the claimed invention, from a study of the drawing, the disclosure, and the appended claims. Moreover, in the drawings and specification, there have been disclosed preferred embodiments and examples of the invention and, although specific terms are employed, they are used in a generic and descriptive sense only and not for the purpose of limitation. The scope of the invention is set forth in the following claims, in which the word ‘comprising’ does not exclude other elements or steps, and the indefinite article ‘a’ or ‘an’ does not exclude a plurality.

### CLAUSES

Clause 1. A method (400) for transmitting information from a medical device (100) to a first server (102, 108), the method comprising:
receiving (S402) first access level data, the first access level data indicating whether the first server is configured to receive personal identifiable information, PII, or not from the medical device;
determining (S404) available information at the medical device;
classifying (S406) the available information into at least one class of a plurality of classes, the plurality of classes comprising PII and non-PII;
upon the first access level data indicating that the first server is not configured to receive PII, filtering (S408) the available information to remove PII and transmitting (S410) the filtered available information from the medical device to the first server; and
upon the first access level data indicating that the first server is configured to receive PII, transmitting (S410) the available information from the medical device to the first server.

Clause 2. The method according to clause 1, wherein the non-PII comprises equipment data pertaining to the medical device.

Clause 3. The method according to any one of clauses 1-2, wherein the PII comprises clinical data pertaining to the medical device.

Clause 4. The method according to any one of clauses 1-3, wherein the medical device and the first server (102) are arranged in a local network (104), wherein the first access level data indicates that the server is configured to receive PII.

Clause 5. The method according to clause 4, further comprising the steps of:
receiving the transmitted information at the first server (102), the transmitted information comprising PII;
receiving (S412) second access level data, the second access level data indicating that a second server (108) is not configured to receive PII; and
filtering (S414) the received information to remove PII and transmitting (S416) the filtered received information from the first server to the second server.

Clause 6. The method of clause 5, wherein the first server and the medical device are arranged in a local network, and wherein the second server is arranged externally to the local network.

Clause 7. The method of any one of clauses 4-6, wherein the first server comprises an environment for running a clinical application using the information received from the medical device.

Clause 8. The method according to any one of clauses 1-3, wherein medical device is arranged in a local network, wherein the first server (108) is arranged externally to the local network, and wherein access level data indicates that the first server is not configured to receive PII.

Clause 9. The method according to any one of clauses 1-8, wherein the step of receiving first access level data comprises performing a handshake procedure between the medical device and the first server.

Clause 10. The method according to any one of clauses 1-8, wherein the step of receiving first access level data comprises:
receiving a certificate associated with the first server;
verifying the certificate to determine validity of the certificate; and
upon determining that the certificate is valid, extracting the first access level data from the certificate.

Clause 11. The method according to any one of clauses 1-10, wherein the first access level data is received upon installation of the medical device.

Clause 12. The method according to any one of clauses 1-11, wherein the medical device is at least one of: a cardiac support device, a respiratory support device, or a or monitoring device.

Clause 13. The method according to any one of clauses 1-12, wherein the medical device comprises a display, and wherein the method further comprises the step of:
displaying the available information on the display.

Clause 14. A medical device (100) comprising:
one or more processors; and
one or more non-transitory computer-readable media storing first computer executable instructions that, when executed by the one or more processors, cause the medical device to perform actions comprising:
receiving (S402) first access level data, the first access level data indicating whether a first server (102, 108) is configured to receive personal identifiable information, PII, or not from the medical device;
determining (S404) available information at the medical device;
classifying (S406) the available information into at least one class of a plurality of classes, the plurality of classes comprising PII and non-PII;
upon the first access level data indicating that the first server is not configured to receive PII, filtering (S408) the available information to remove PII and transmitting (S410) the filtered available information to the first server; and
upon the first access level data indicating that the first server is configured to receive PII, transmitting (S410) the available information to the first server.

Clause 15. One or more non-transitory computer-readable media storing instructions executable by one or more processors, wherein the instructions, when executed, cause the one or more processors to perform operations comprising:
receiving (S402) first access level data, the first access level data indicating whether a first server (102, 108) is configured to receive personal identifiable information, PII, or not from a medical device (100);
determining (S404) available information at the medical device;
classifying (S406) the available information into at least one class of a plurality of classes, the plurality of classes comprising PII and non-PII;
upon the first access level data indicating that the first server is not configured to receive PII, filtering (S408) the available information to remove PII and transmitting (S410) the filtered available information to the first server, and
upon the first access level data indicating that the first server is configured to receive PII, transmitting (S410) the available information to the first server.

1. A method for transmitting information from a medical device to a first server, the method comprising:
receiving first access level data, the first access level data indicating whether the first server is configured to receive personal identifiable information, PII, or not from the medical device;
determining available information at the medical device;
classifying the available information into at least one class of a plurality of classes, the plurality of classes comprising PII and non-PII;
upon the first access level data indicating that the first server is not configured to receive PII, filtering the available information to remove PII and transmitting the filtered available information from the medical device to the first server; and
upon the first access level data indicating that the first server is configured to receive PII, transmitting the available information from the medical device to the first server.

2. The method according to claim 1, wherein the non-PII comprises equipment data pertaining to the medical device.

3. The method according to claim 1, wherein the PII comprises clinical data pertaining to the medical device.

4. The method according to claim 1, wherein the medical device and the first server are arranged in a local network, wherein the first access level data indicates that the server is configured to receive PII.

5. The method according to claim 4, further comprising the steps of:
receiving the transmitted information at the first server, the transmitted information comprising PII;
receiving second access level data, the second access level data indicating that a second server is not configured to receive PII; and
filtering the received information to remove PII and transmitting the filtered received information from the first server to the second server.

6. The method of claim 5, wherein the first server and the medical device are arranged in a local network, and wherein the second server is arranged externally to the local network.

7. The method of claim 4, wherein the first server comprises an environment for running a clinical application using the information received from the medical device.

8. The method according to claim 1, wherein medical device is arranged in a local network, wherein the first server is arranged externally to the local network, and wherein access level data indicates that the first server is not configured to receive PII.

9. The method according to claim 1, wherein the step of receiving first access level data comprises performing a handshake procedure between the medical device and the first server.

10. The method according to claim 1, wherein the step of receiving first access level data comprises:
receiving a certificate associated with the first server;
verifying the certificate to determine validity of the certificate; and
upon determining that the certificate is valid, extracting the first access level data from the certificate.

11. The method according to claim 1, wherein the first access level data is received upon installation of the medical device.

12. The method according to claim 1, wherein the medical device is at least one of: a cardiac support device, a respiratory support device, or a or monitoring device.

13. The method according to claim 1, wherein the medical device comprises a display, and wherein the method further comprises the step of:
displaying the available information on the display.

14. A medical device comprising:
one or more processors; and
one or more non-transitory computer-readable media storing first computer executable instructions that, when executed by the one or more processors, cause the medical device to perform actions comprising:
receiving first access level data, the first access level data indicating whether a first server is configured to receive personal identifiable information, PII, or not from the medical device;
determining available information at the medical device;
classifying the available information into at least one class of a plurality of classes, the plurality of classes comprising PII and non-PII;
upon the first access level data indicating that the first server is not configured to receive PII, filtering the available information to remove PII and transmitting the filtered available information to the first server; and
upon the first access level data indicating that the first server is configured to receive PII, transmitting the available information to the first server.

15. One or more non-transitory computer-readable media storing instructions executable by one or more processors, wherein the instructions, when executed, cause the one or more processors to perform operations comprising:
receiving first access level data, the first access level data indicating whether a first server is configured to receive personal identifiable information, PII, or not from a medical device;
determining available information at the medical device;
classifying the available information into at least one class of a plurality of classes, the plurality of classes comprising PII and non-PII;
upon the first access level data indicating that the first server is not configured to receive PII, filtering the available information to remove PII and transmitting the filtered available information to the first server; and
upon the first access level data indicating that the first server is configured to receive PII, transmitting the available information to the first server.
